(19) United States
(12) Patent Application Publication
(10) Pub. No.: US 2024/0165403 A1
(43) Pub. Date: May 23, 2024

(54) ANCHOR

(71) Applicant: GIMER MEDICAL. Co. LTD., New Taipei City (TW)

(72) Inventors: Zh Huan YING, New Taipei City (TW); Chen-Tun WU, New Taipei City (TW)

(73) Assignee: GIMER MEDICAL. Co. LTD., New Taipei City (TW)

(21) Appl. No.: 18/513,000

(22) Filed: Nov. 17, 2023

(30) Foreign Application Priority Data
Nov. 18, 2022 (CN) 202211447714.8

(51) Int. Cl. A61N 1/05

(52) U.S. Cl. CPC A61N 1/0558

(57) ABSTRACT

An anchor for a lead is provided, including a protecting member, a fixed member, and a rotating member. The protecting member has an accommodating slot, a first through hole, and a second through hole. The accommodating slot is disposed between the first through hole and the second through hole, and is communicated with the first through hole and the second through hole. The fixed member is disposed in the accommodating slot and includes a depression structure. The rotating member is pivotally connected to the fixed member, and includes a contacting portion. When the lead passes through the first through hole, the depression structure, and the second through hole, the rotating member is enabled to rotate relative to the fixed member to let the contacting portion be accommodated in the depression structure and compress the lead.

### CROSS REFERENCE TO RELATED APPLICATIONS

The present application claims priority of China Patent Application No. 202211447714.8, filed Nov. 18, 2022, which is incorporated by reference herein in its entirety.

### BACKGROUND

Technology Field The application relates in general to an anchor, and in particular, to the anchor of an electrical stimulation device.

### Description of the Related Art

Spinal cord stimulation (SCS), dorsal column stimulation (DCS) and peripheral nerve stimulation (PNS) use implanted electrical stimulation devices or non-implanted electrical stimulation devices to transmit electrical signals to the target region of a user's spinal cord (or their dorsal column) for pain relief. The electrical stimulation device usually includes a pulse generator, a lead with at least one electrodes. The pulse generator creates an electrical signal, and transmits the electrical signal to the target region by the electrodes. The lead with the electrode is also at least partially implanted into the tissue.

In general, the lead is used in conjunction with an anchor. The anchor is configured to affix the lead to the tissue, and avoids the migration of the lead from the target location thus affecting the therapeutic effect. However, an additional tool is required to affix anchors which are currently in use, which can be inconvenient. Therefore, how to address the aforementioned problem has become an important issue.

### SUMMARY

To address the deficiencies of conventional products, an embodiment of the disclosure provides an anchor for fixing a lead in the tissue, including a protecting member, a fixed member, a rotating member, and a hinge. The protecting member has an accommodating slot, a first through hole, and a second through hole. The accommodating slot is disposed between the first through hole and the second through hole, and is communicated with the first through hole and the second through hole. The fixed member is disposed in the accommodating slot and includes a depression structure. The rotating member includes a contacting portion. The hinge is pivotally connected to the fixed member and the rotating member. When the rotating member is at an opened angle relative to the fixed member, at least a portion of the contacting portion is disposed outside the depression structure. When the rotating member rotates from the opened angle to a closed angle, the contacting portion is accommodated in the depression structure.

In some embodiments, the depression structure has a first inner surface, a second inner surface, and an opening, the first inner surface faces the second inner surface, and the width of the opening is less than the maximum distance between the first inner surface and the second inner surface. The maximum width of the contacting portion is greater than the width of the opening.

In some embodiments, the depression structure has a bottom surface, and when the rotating member is at a closed angle, the distance between the contacting portion and the bottom surface is less than the diameter of the lead.

In some embodiments, the depression structure has a bottom surface. When the rotating member is at the closed angle, the distance between the contacting portion and the bottom surface is less than the diameter of the lead.

In some embodiments, the fixed member has a base, a first lateral wall, and a second lateral wall, the first lateral wall and the second lateral wall are connected to the base, and the depression structure is formed between the first lateral wall and the second lateral wall, wherein the first lateral wall and the second lateral wall are elastic.

In some embodiments, the fixed member further has a pivot portion, the pivot portion is connected to the base, and the hinge is pivotally connected to the pivot portion and the rotating member, wherein a gap is formed between the pivot portion and the first lateral wall.

In some embodiments, the fixed member comprises an abutting portion, connected to the first lateral wall and having an abutting surface, wherein an angle is formed between the first lateral wall and the abutting surface. The protecting member and the abutting surface are separable.

In some embodiments, the diameter of the first through hole is greater than the diameter of the lead, and the diameter of the second through hole is greater than the diameter of the lead.

In some embodiments, the protecting member has an outer surface, and when the rotating member is at the closed angle, the rotating member does not protrude from the outer surface. In some embodiments, a plurality of notches parallel to each other are formed on the outer surface.

In some embodiments, the material of the protecting member is different from the material of the fixed member.

### BRIEF DESCRIPTION OF DRAWINGS

The disclosure can be more fully understood by reading the subsequent detailed description and examples with references made to the accompanying drawings, wherein:

FIG. 1 is a schematic diagram of an electrical stimulation device disposed in a human body according to an embodiment of the invention;

FIG. 2 is a schematic diagram of an anchor according to an embodiment of the disclosure;

FIG. 3 is a exploded-view diagram of the anchor according to an embodiment of the disclosure;

FIG. 4 is a cross-sectional view taken along the line A-A in FIG. 2;

FIG. 5 is a cross-sectional view taken along the line B-B in FIG. 2;

FIG. 6A is a schematic diagram representing that a rotating member of the anchor is at an opened angle relative to a fixed member according to an embodiment of the disclosure;

FIG. 6B is a cross-sectional view taken along the line C-C in FIG. 6A;

FIG. 6C is a schematic diagram representing that a lead passes through the anchor according to an embodiment of the disclosure;

FIG. 6D is a schematic diagram representing that the rotating member of the

anchor rotates from the opened angle to a closed angle relative to the fixed member according to an embodiment of the disclosure;

FIG. 6E is a cross-sectional view taken along the line D-D in FIG. 6D; and

FIG. 6F is a schematic diagram representing that the rotating member of the anchor rotates from the closed angle to the opened angle relative to the fixed member by using a plate according to an embodiment of the disclosure.

### DETAILED DESCRIPTION OF DISCLOSURE

The making and using of the embodiments of the anchor are discussed in detail below. It should be appreciated, however, that the embodiments provide many applicable inventive concepts that can be embodied in a wide variety of specific contexts. The specific embodiments discussed are merely illustrative of specific ways to make and use the embodiments, and do not limit the scope of the disclosure.

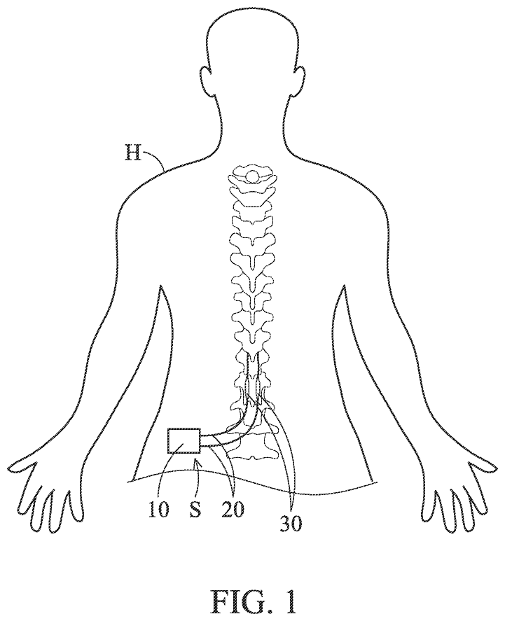

Referring to FIG. 1, an implanted treating device S can be implanted into a human body H, and can measure the physiological signal of the human body or treat the affected part of the human body H. The implanted treating device S can include an implanted controller 10, at least one lead 20, and at least one anchor 30. The lead 20 is electrically connected to the implanted controller 10. The anchor 30 is connected to the lead 20 and affixed in the human body H, so as to position the lead 20 to a required position. It should be noted that, although the embodiment takes the implanted treating device S as an example, the disclosure is not limited thereto. For example, the disclosure can also be used on a non-implanted treating device, that is, the controller is arranged outside the human body, and at least one and at least one anchor are implanted in the human body.

The opposite ends of the lead 20 are respectively a proximal end a distal end, having a plurality of electrodes (not shown). The plurality of electrodes at the proximal end of the lead 20 are electrically connected to the implanted controller 10, and the anchor 30 is used to fix the lead in the tissue for positioning the plurality of electrodes at the distal end of the lead 20 to the desired tissue region of the human body. In this embodiment, the implanted treating device S is an implanted electrical stimulation device, and the implanted controller 10 includes a pulse generator. The pulse generator is configured to create the electric signal, and the electrical signal is transmitted to the target region by the lead and the plurality of electrodes.

For example, the anchor 30 can be affixed to the fascia, the interspinous ligament, or the supraspinous ligament under the surgical wound of the implantation, and the lead 20 can be positioned on the epidural space of the human body H, but it is not limited thereto. The implanted treating device S can be used in the surgery of the spinal cord stimulation (SCS). When the implanted treating device S is implanted into the tissue of human body H, the user can remotely active the implanted controller 10, and the implanted controller 10 can provide the electric signal (such as the current) to the lead 20. Thus, the lead 20 can stimulate the nerves of the spinal cord, and the pain of the affected part of the human body H can be reduced.

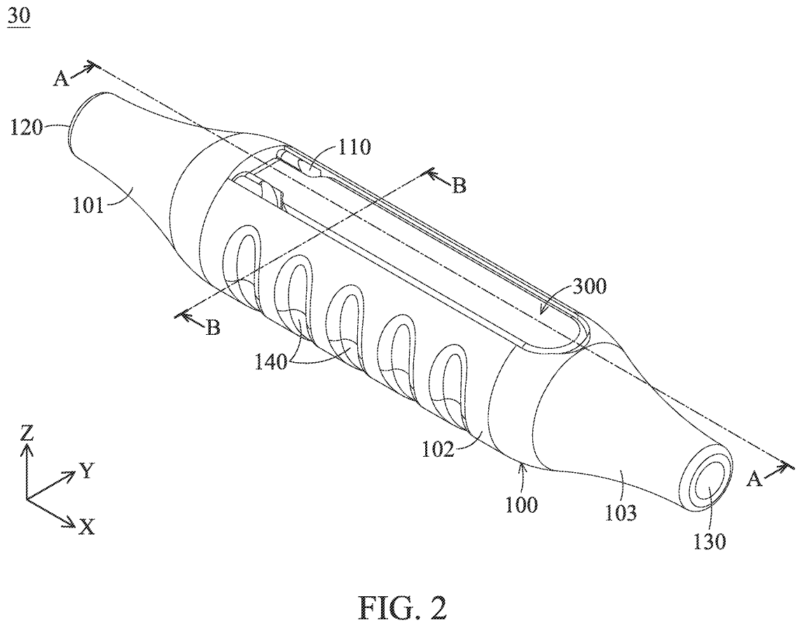

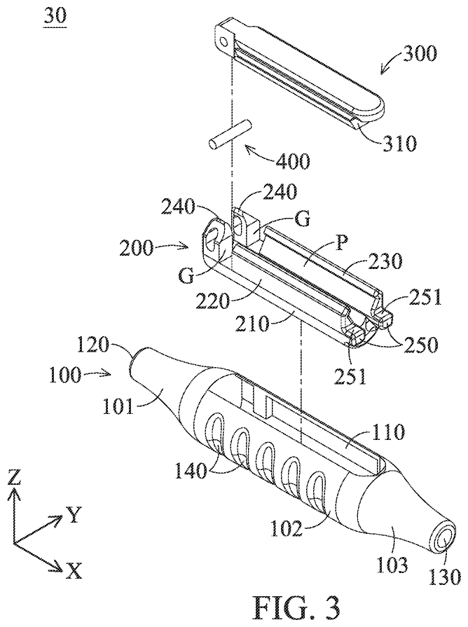

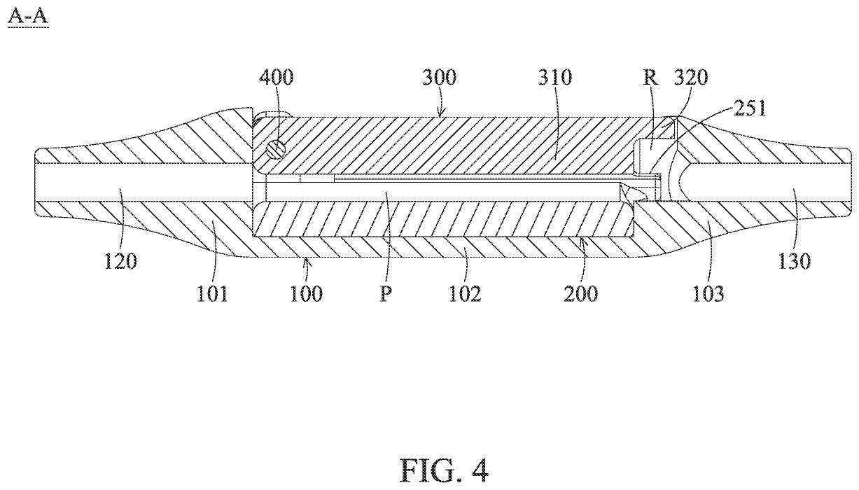

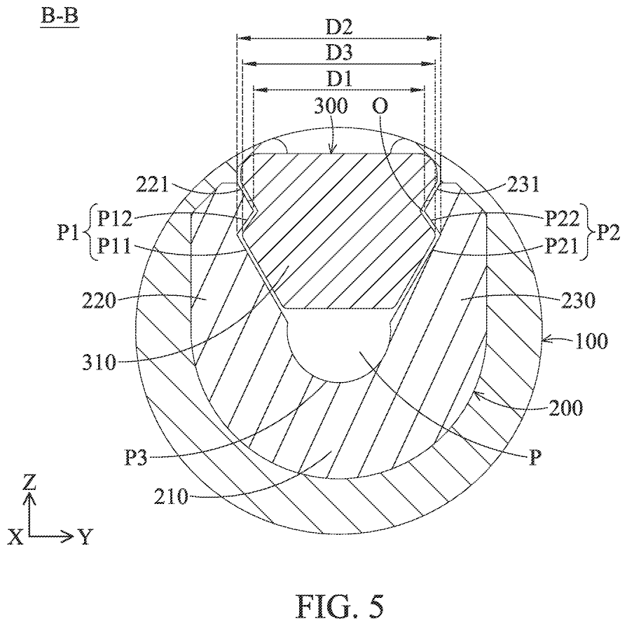

FIG. 2 and FIG. 3 are a schematic diagram and a exploded-view diagram of the anchor 30 respectively, and FIG. 4 and FIG. 5 are a cross-sectional view taken along the line A-A in FIG. 2 and a cross-sectional view taken along the line B-B in FIG. 2 respectively. As shown in FIG. 2 to FIG. 5, the anchor 30 primarily includes a protecting member 100, a fixed member 200, a rotating member 300, and a hinge 400. The structure and the connection relationship of the aforementioned members are discussed below.

The protecting member 100 can be divided into three sections, including a first section 101, a second section 102 and a third section 103. The second section 102 substantially has a cylindrical structure, and each of the first section 101 and the third section 103 substantially has an outward taper structure. The second section 102 is disposed between the first section 101 and the third section 103. The protecting member 100 has an accommodating slot 110 formed on the second section 102, a first through hole 120 formed on the first section 101, and a second through hole 130 formed on the third section 103. The first through hole 120 is extended along an arrangement direction of the first section 101, the second section 102, and the third section 103 (the X-axis), penetrates the first section 101, and is communicated with the accommodating slot 110 on the second section 102. Similarly, the second through hole 130 is extended along the arrangement direction of the first section 101, the second section 102, and the third section 103 (the X-axis), penetrates the third section 103, and is communicated with the accommodating slot 110 on the second section 102.

In this embodiment, the diameter of the first through hole 120 and the diameter of the second through hole 130 is greater than the diameter of the lead 20. As observed along the X-axis, the first through hole 120 is substantially aligned with the second through hole 130. Therefore, it is facilitated for the user to pass the lead 20 through the first through hole 120 and the second through hole 130. Moreover, in this embodiment, a plurality of notches 140 parallel to each other are formed on the second section 102 of the protecting member 100.

The protecting member 100 can be made of a cushioning material that is flexible and able to prevent the human body H from experiencing a rejection reaction. For example, the protecting member 100 can include silicone, but it is not limited thereto.

Referring to FIG. 2 to FIG. 5, the fixed member 200 can be disposed in the accommodating slot 110 of the protecting member 100. The fixed member 200 includes a base 210, a first lateral wall 220, a second lateral wall 230, at least one pivot portion 240, and at least one abutting portion 250. The first lateral wall 220 and the second lateral wall 230 are adjacent to the base 210, and the first lateral wall 220 are spaced from the second lateral wall 230. Therefore, a depression structure P can be formed by the first lateral wall 220, the second lateral wall 230, and the base 210. When the fixed member 200 is disposed in the accommodating slot 110, the first through hole 120 and the second through hole 130 are communicated with the depression structure P.

The surface of the first lateral wall 220 facing the second lateral wall 230 forms a first inner surface P1 of the depression structure P, the surface of the second lateral wall 230 facing the first lateral wall 220 forms a second inner surface P2 of the depression structure P, and the top surface of the base 210 forms a bottom surface P3 of the depression structure P. In this embodiment, the bottom surface P can be a curved surface, so as to facilitate the disposition of the lead 20.

It should be noted that, the first inner surface P1 and the second inner surface P2 can be convex. In detail, the first inner surface P1 includes a surface P11 and a surface P12. The surface P12 is connected to the surface P11, the surface P11 is extended from the connection point of the surface P11 and the bottom surface P3 to the connection point of the surface P11 and the surface P12 along a direction away from the second lateral wall 230, and the surface P12 is extended from the connection point of the surface P11 and the surface P12 toward the second lateral wall 230. Similarly, the second inner surface P2 includes a surface P21 and a surface P22. The surface P22 is adjacent to the surface P21, the surface P21 is extended from the connection point of the surface P21 and the bottom surface P3 to the connection point of the surface P21 and the surface P22 along a direction away from the first lateral wall 220, and the surface P22 is extended from the connection point of the surface P21 and the surface P22 toward the first lateral wall 220. It should be noted that the width D1 of the opening O in the depression structure P is less than the maximum distance D2 between the first inner surface P1 and the second inner surface P2.

In this embodiment, the first lateral wall 220 further includes a guiding surface 221, and the second lateral wall 230 further includes a guiding surface 231. The guiding surface 221 of the first lateral wall 220 is connected to the surface P12, and is extended from the connection point of the guiding surface 221 and the surface P12 along the direction away from the second lateral wall 230. The guiding surface 231 of the second lateral wall 230 is adjacent to the surface P22, and is extended from the connection point of the guiding surface 231 and the surface P22 along the direction away from the first lateral wall 220.

The pivot portion 240 and the abutting portion 250 are adjacent to the base 210, and respectively adjacent to the first section 101 and the third section 103 of the protecting member 100. Therefore, the first lateral wall 220 and the second lateral wall 230 are located between the pivot portion 240 and the abutting portion 250. Specifically, the pivot portion 240 is separated from the first lateral wall 220 and the second lateral wall 230. That is, a gap G is formed between the pivot portion 240 and the first lateral wall 220, and another gap G is formed between the pivot portion 240 and the second lateral wall 230. The abutting portion 250 is directly connected to the first lateral wall 220 and the second lateral wall 230, and the first lateral wall 220 and the second lateral wall 230 protrude from the abutting surface 251 of the abutting portion 250. Thus, an angle can be formed between the abutting surface 251 and the first lateral wall 220, and another angle can be formed between the abutting surface 251 and the second lateral wall 230. In this embodiment, the angle between the abutting surface 251 and the first lateral wall 220 and the angle between the abutting surface 251 and the second lateral wall 230 are 90 degrees, but it is not limited thereto.

When the fixed member 200 is disposed in the accommodating slot 110, the base 210, the first lateral wall 220, the second lateral wall 230, and the pivot portion 240 are affixed to the protecting member 100 by an adhesive glue, and the abutting portion 250 does not adhere to the protecting member 100.

The rotating member 300 can be pivotally connected to the pivot portion 240 of the fixed member 200 via the hinge 400. The rotating member 300 includes a contacting portion 310. In this embodiment, the appearance and the dimension of the contacting portion 310 correspond to the first inner surface P1 and the second inner surface P2. Therefore, a maximum width D3 of the contacting portion 310 is greater than the width D1 of the opening O of the depression structure P. In this embodiment, the maximum width D3 of the contacting portion 310 is slightly less than the maximum distance D2 between the first inner surface P1 and the second inner surface P2.

Moreover, as shown in FIG. 4, the rotating member 300 can include an extending portion 320. The arrangement of the extending portion 320 corresponds to the abutting surface 251, and the extending portion 320 is spaced away from the abutting surface 251. Therefore, owing to the extending portion 320 of the rotating member 300 and the abutting surface 251 of the fixed member 200, a recess R can be formed between the rotating member 300 and the fixed member 200. The recess R can facilitate the user to operate the rotating member 300.

The fixed member 200 and the rotating member 300 can be made of a cushioning material that is elastic and has sufficient hardness. The hardness of the fixed member 200 and the rotating member 300 can be greater than the hardness of the protecting member 100. For example, the material of the fixed member 200 and the rotating member 300 can include polyetheretherketone (PEEK), but it is not limited thereto.

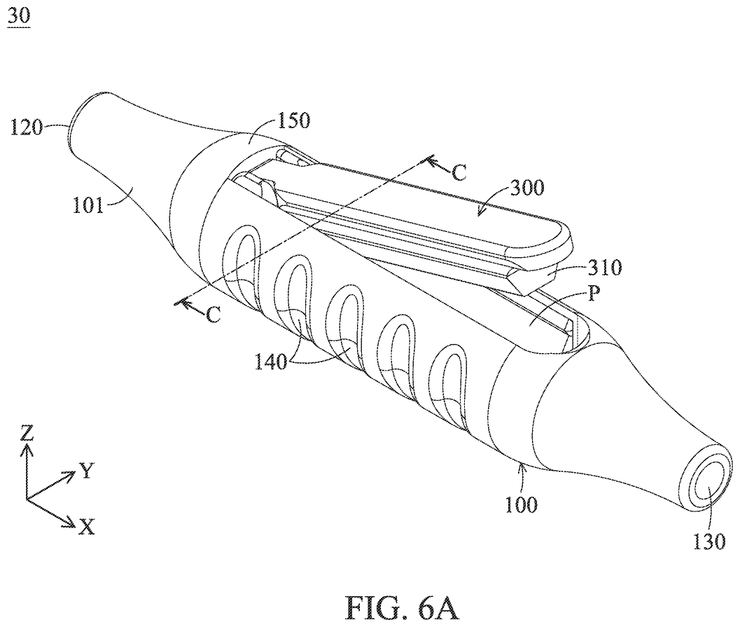

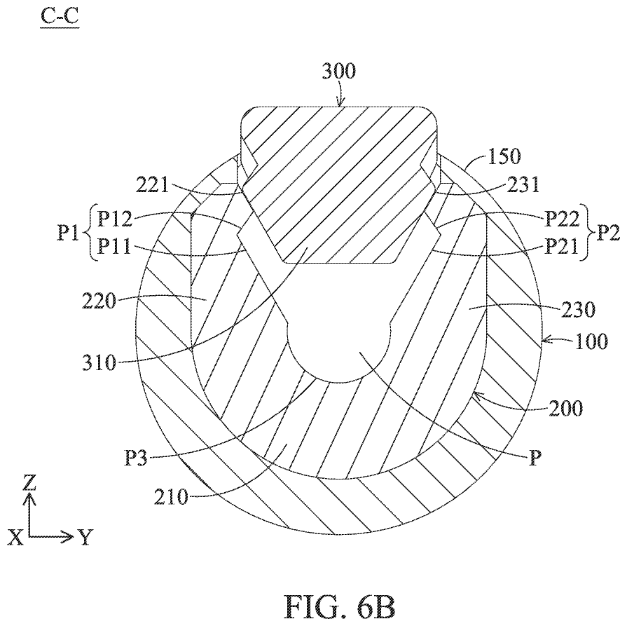

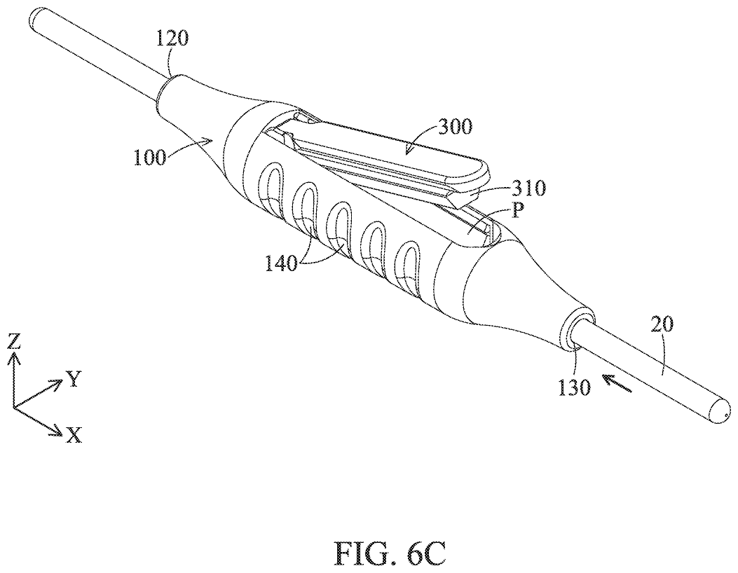

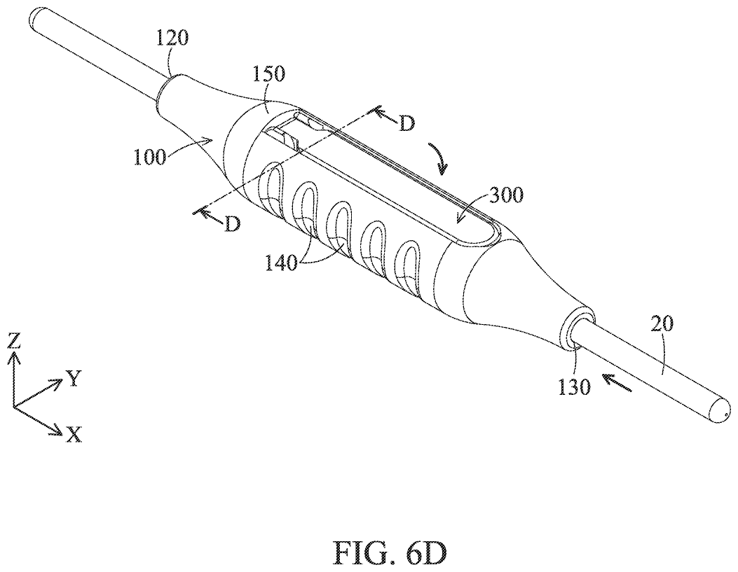

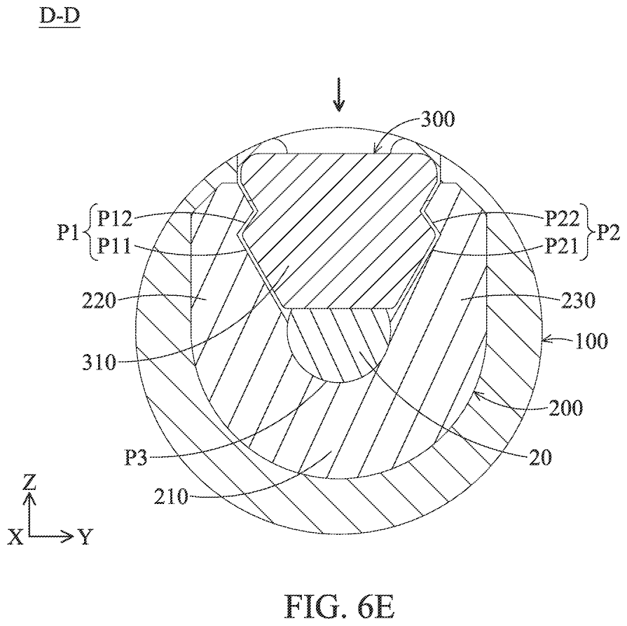

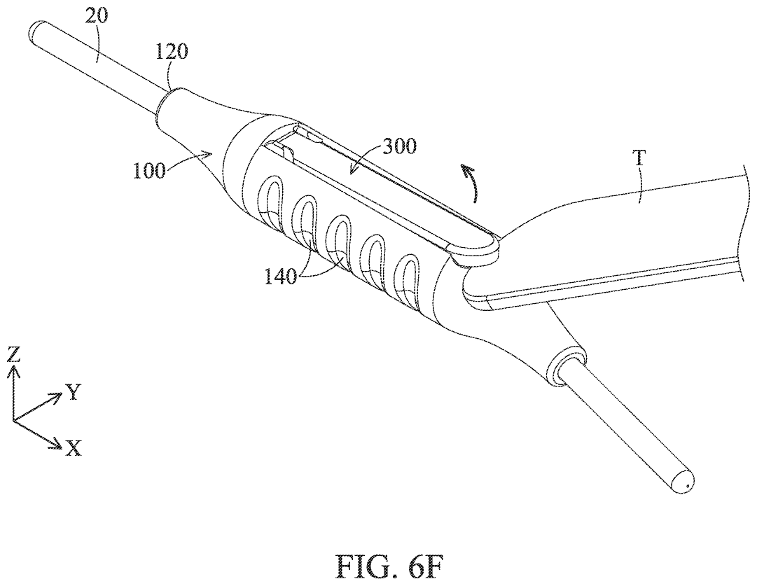

The usage of the anchor 30 is discussed below. Referring to FIG. 6A, and FIG. 6B, when the rotating member 300 is at an opened angle relative to the fixed member 200, the rotating member 300 protrudes relative to the outer surface 150 of the protecting member 100. Thus, at least a portion of the contacting portion 310 is disposed outside the depression structure P of the fixed member 200.

As shown in FIG. 6C, when the rotating member 300 is at the opened angle, the user can pass the lead 20 through the second through hole 130, the depression structure P, and the first through hole 120. Since the diameter of the first through hole and the diameter of the second through hole 130 are greater than the diameter of the lead 20, the lead 20 can directly pass through the anchor 30 without the assistant of the extra tool when the user applies the aforementioned operation.

Subsequently, referring to FIG. 6D and FIG. 6E, the user can apply force to the rotating member 300 (for example, the user can push the rotating member 300 with a thumb), so that the rotating member 300 can rotate from the opened angle to the closed angle. Since the first lateral wall 220 and the second lateral wall 230 of the fixed member 200 are elastic, the rotating member 300 can push the first lateral wall 220 and the second lateral wall 230 along the guiding surfaces 221 and 231, and the first lateral wall 220 and the second lateral wall 230 are slightly deformed to let the contacting portion 310 enter the depression structure P. When the position of the contacting portion 310 corresponds to the positions of the first inner surface P1 and the second inner surface P2 (i.e. when the rotating member 300 is at the closed angle relative to the fixed member 200), the first lateral wall 220 and the second lateral wall 230 can return to the non-deformed state.

In this embodiment, since the fixed member 200 has two gaps G shown in FIG. 3, two pivot portions 240 are not influenced by the deformation of the first lateral wall 220 and the second lateral wall 230. Therefore, the unsmooth in the rotation of the rotating member 300 can be prevented.

In this embodiment, a structure corresponding to the first lateral wall 220, the second lateral wall 230, and the gap G can be disposed at the inner side of the protecting member 100. For example, the protecting member 100 can include rib corresponding to the position of the gap G to embed into the gap G, but it is not limited thereto.

As shown in FIG. 6D and FIG. 6E, when the rotating member 300 is at the closed angle relative to the fixed member 200, the distance between the contacting portion 310 and the bottom surface P3 of the depression structure P is less than the diameter of the lead 20. Therefore, the contacting portion 310 is in contact with the lead 20 and compresses the lead 20. The lead 20 is slightly deformed, so that it can be clamped and affixed in the depression structure P. Accordingly, the lead 20 does not displace in the anchor 30. Moreover, since the width D1 of the opening O is less than the maximum width D3 of the contacting portion 310, the resilience of the lead 20 is not sufficient to push out the contact portion 310 from the depression structure P, and the rotating member 300 can be positioned in the closed angle relative to the fixed member 200. In detail, the width D1 of the opening O of the depression structure P is the smallest width of the opening O, or the fastening width of the depression structure P is less than the fastening width of the rotating member 300. Thus, the resilience of the lead 20 is not sufficient to push out the contact portion 310 from the depression structure P when there is no external force.

In this embodiment, when the rotating member 300 is at the closed angle relative to the fixed member 200, the rotating member 300 does not protrude from the outer surface 150 of the protecting member 100. The anchor 30 can achieve an integrated appearance, and the discomfort when the anchor 30 is implanted in the human body H can be reduced.

When the lead 20 is affixed to the anchor 30 by the aforementioned steps, the user can use the suture to affix the anchor 30 to the fascia, the interspinous ligament, or the supraspinous ligament. The suture can wind around the notches 140 of the anchor 30 to enhance the affixing effect of the anchor 30.

Referring to FIG. 6F, if the user wants to adjust the relative position of the lead 20 and the anchor 30, the protecting member 100 can be separated from the abutting portion 250, and a plate T ban be inserted into the recess R between the rotating member 300 and the fixed member 200, wherein the plate T can be adjacent to the abutting surface 251 of the abutting portion 250, the first lateral wall 220, and the second lateral wall 230. When the plate T rotates toward the rotating member 300, the plate T can push the extending portion 320, the rotating member 300 can therefor rotate from the closed angle to the opened angle, and the lead 20 is enabled to move relative to the anchor 30.

In some embodiments, the abutting portion 250 is directly adhered to the protecting member 100, and the protecting member 100 has a high deformation degree. Thus, the plate T can abut the abutting surface 251 through the protecting member 100.

In summary, an embodiment of the disclosure provides an anchor for fixing a lead in the tissue, including a protecting member, a fixed member, a rotating member, and a hinge. The protecting member has an accommodating slot, a first through hole, and a second through hole. The accommodating slot is disposed between the first through hole and the second through hole, and is communicated with the first through hole and the second through hole. The fixed member is disposed in the accommodating slot and includes a depression structure. The rotating member includes a contacting portion. The hinge is pivotally connected to the fixed member and the rotating member. When the rotating member is at an opened angle relative to the fixed member, at least a portion of the contacting portion is disposed outside the depression structure. When the rotating member rotates from the opened angle to a closed angle, the contacting portion is accommodated in the depression structure.

Although some embodiments of the present disclosure and their advantages have been described in detail, it should be understood that various changes, substitutions and alterations can be made herein without departing from the spirit and scope of the disclosure as defined by the appended claims. For example, it will be readily understood by those skilled in the art that many of the features, functions, processes, and materials described herein may be varied while remaining within the scope of the present disclosure. Moreover, the scope of the present application is not intended to be limited to the particular embodiments of the process, machine, manufacture, compositions of matter, means, methods and steps described in the specification. As one of ordinary skill in the art will readily appreciate from the disclosure of the present disclosure, processes, machines, manufacture, compositions of matter, means, methods, or steps, presently existing or later to be developed, that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps. Moreover, the scope of the appended claims should be accorded the broadest interpretation so as to encompass all such modifications and similar arrangements.

While the disclosure has been described by way of example and in terms of preferred embodiment, it should be understood that the disclosure is not limited thereto. On the contrary, it is intended to cover various modifications and similar arrangements (as would be apparent to those skilled in the art). Therefore, the scope of the appended claims should be accorded the broadest interpretation to encompass all such modifications and similar arrangements.

What is claimed is:

1. An anchor for a lead, comprising:
a protecting member, having an accommodating slot, a first through hole, and a second through hole, wherein the accommodating slot is disposed between the first through hole and the second through hole, and the accommodating slot is communicated with the first through hole and the second through hole;
a fixed member, disposed in the accommodating slot and having a depression structure; and
a rotating member, comprising a contacting portion and pivotally connected to the fixed member, wherein when the lead passes through the first through hole, the depression structure, and the second through hole, the rotating member is enabled to rotate relative to the fixed member to let the contacting portion be accommodated in the depression structure and compress the lead.

2. The anchor as claimed in claim 1, wherein the depression structure has a first inner surface, a second inner surface, and an opening, the first inner surface faces the second inner surface, and a width of the opening is less than a maximum distance between the first inner surface and the second inner surface.

3. The anchor as claimed in claim 2, wherein a maximum width of the contacting portion is greater than the width of the opening.

4. The anchor as claimed in claim 1, wherein the depression structure has a bottom surface, and when the rotating member is at a closed angle, a distance between the contacting portion and the bottom surface is less than a diameter of the lead.

5. The anchor as claimed in claim 1, wherein the fixed member has a base, a first lateral wall, and a second lateral wall, the first lateral wall and the second lateral wall are adjacent to the base, and the depression structure is formed between the first lateral wall and the second lateral wall, wherein the first lateral wall and the second lateral wall are elastic.

6. The anchor as claimed in claim 5, wherein the fixed member further has a pivot portion, the pivot portion is connected to the base, and a hinge is pivotally connected to the pivot portion and the rotating member, wherein a gap is formed between the pivot portion and the first lateral wall, and an additional gap is formed between the pivot portion and the second lateral wall.

7. The anchor as claimed in claim 5, wherein the fixed member comprises an abutting portion, adjacent to the first lateral wall and having an abutting surface, wherein an angle is formed between the first lateral wall and the abutting surface.

8. The anchor as claimed in claim 7, wherein the protecting member and the abutting surface are separable.

9. The anchor as claimed in claim 1, wherein a diameter of the first through hole is greater than a diameter of the lead, and a diameter of the second through hole is greater than the diameter of the lead.

10. The anchor as claimed in claim 1, wherein the protecting member has an outer surface, and when the rotating member is at a closed angle, the rotating member does not protrude from the outer surface.

11. The anchor as claimed in claim 1, wherein the protecting member has an outer surface, and a plurality of notches parallel to each other are formed on the outer surface.

12. The anchor as claimed in claim 1, wherein a hardness of the protecting member is less than a hardness of the fixed member.

13. The anchor as claimed in claim 1, wherein the anchor further comprises a hinge, pivotally connected to the fixed member and the rotating member.

14. The anchor as claimed in claim 1, wherein when the rotating member is in an opened angle relative to the fixed member, at least a portion of the contacting portion is disposed outside the depression structure, wherein when the rotating member rotates from the opened angle to a closed angle, the contacting portion is accommodated in the depression structure.
